(19) United States
(12) Patent Application Publication
(10) Pub. No.: US 2007/0055098 A1
(43) Pub. Date: Mar. 8, 2007

(54) In-body information acquisition system

(75) Inventors: Hatsuo Shimizu, Tokyo (JP); Mikio Nakamura, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Corporation, Tokyo (JP)

(21) Appl. No.: 11/515,574

(22) Filed: Sep. 5, 2006

(30) Foreign Application Priority Data
Sep. 5, 2005 (JP) 2005-256262

(51) Int. Cl. A61B 1/00; A61B 1/04

(52) U.S. Cl. 600/109; 600/160; 600/118

(57) ABSTRACT

An in-body information acquisition system includes a medical capsule device which is introduced inside a body, and an apparatus outside the body which is disposed outside the body, and which communicates with the medical capsule device. The medical capsule device includes at least a first pad, and the apparatus outside the body includes at least a second pad. For transceiving a signal between the first pad and the second pad, at least one of the medical capsule device and the apparatus outside the body includes a modulating unit which modulates a signal, and applies a voltage to the pad of one of the medical capsule device and the apparatus outside the body, and the other apparatus includes a demodulating unit which demodulates the signal based on a change in an electric potential of the pad of the other apparatus.

### CROSS-REFERENCE TO RELATED APPLICATION

The present application is based upon and claims the benefit of priority from the prior Japanese Patent Application No. 2005-256262 filed on Sep. 5, 2005; the entire contents of which are incorporated herein by reference.

### BACKGROUND OF THE INVENTION

1. Field of the Invention

The present invention relates to an in-body information acquisition system in which information of inside of a body examined is communicated between an apparatus which is disposed inside the body, and an apparatus which is disposed outside the body.

2. Description of the Related Art

In recent years, in a field of body to be examined, particularly in vivo examination, and treatment, in vivo information which is acquired in vivo or near the living body is required to be communicated outside the living body. For communicating the information to the outside, a structure for electric wave communication has been proposed (for example refer to Japanese Patent Application Laid-open Publication No. 2004-524076). In Japanese Patent Application Laid-open Publication No. 2004-524076, a system which includes an apparatus including a transmitter and an in vivo sensor inserted in vivo for acquiring the in vivo information, and a receiver which receives the in vivo information, has been disclosed. Moreover, the information is exchanged with the outside of the body by performing a wireless transmission or an electric wave transmission by a transmitter.

Moreover, a structure which performs communication by allowing a weak current to flow in the living body for transmitting the in vivo information outside, has been proposed (for example refer to Japanese Patent No. 3376462). An apparatus disclosed in Japanese Patent No. 3376462 includes a modulation-current generating means which allows to flow in the organism a weak modulation current which is modulated by superimposing a signal on a carrier. Furthermore, a receiving section which is disposed in vitro and/or in vivo is structured to receive the weak modulation current via an electrode on a receiving side out of two electrodes.

However, in the structure for electric wave communication between the inside of the living body (in vivo) and the outside of the living body (in vitro) disclosed in Japanese Patent Application Laid-open Publication No. 2004-524076, the following problems (1), (2), and (3) are involved, and there is a substantial strain on (a body of) a patient.

(1) Due to regulations, there is a limitation on a frequency which can be used, and a frequency appropriate for communication between the inside of the living body and the outside of the living body cannot be selected voluntarily.

(2) It is necessary to install an antenna inside and outside the living body for transceiving (i.e. transmitting and/or receiving). Moreover, since the electric waves are attenuated in the living body, a plurality of large scale antennas is required to be installed outside the living body. This results in a substantial strain on the patient.

(3) Furthermore, considering the attenuation etc. of the electric waves, a high electric wave output is necessary. Therefore, there is an increase in a size of units to be disposed in vivo and in vitro, which leads to a substantial strain on the patient.

Moreover, even while performing the communication by allowing the weak current to flow in the living body as in the structure described in Japanese Patent No. 3376462, for detecting and demodulating the weak current, there is an increase in the size of a unit on the receiving side. Therefore, there is a substantial strain on (the living body of) the patient.

### SUMMARY OF THE INVENTION

The present invention is made in view of the abovementioned issues, and it is an object of the present invention to provide in-body information acquisition system which reduces a strain on (a living body of) a patient, as there is no need to increase a size of an apparatus introduced inside the living body due to installing an antenna.

To solve the problems mentioned above, and to attain the object, according to the present invention, there is provided an in-body information acquisition system which includes

an apparatus introduced inside a body which is introduced inside the body, and

an apparatus outside the body which is disposed outside the body, and which communicates with the apparatus introduced inside the body, and

the apparatus introduced inside the body includes at least a first pad, and

the apparatus outside the body includes at least a second pad, and

for transceiving a signal between the first pad and the second pad, at least one of the apparatus introduced inside the body and the apparatus outside the body includes a modulating unit which modulates a signal and applies a voltage to the pad of one of the apparatus introduced inside the body and the apparatus outside the body, and

the other apparatus includes a demodulating unit which demodulates the signal based on a change in an electric potential of the pad of the other apparatus.

According to a preferable aspect of the present invention, it is desirable that the apparatus introduced inside the body has an imaging section which takes an image of a part of the body to be examined, and outputs an image signal, and the apparatus outside the body demodulates the image signal.

According to another preferable aspect of the present invention, it is desirable that the second pad in the apparatus outside the body is disposed to be in contact with a surface of the body.

Moreover, according to still another aspect of the present invention, it is desirable that an insulating member is provided on at least one of the first pad and the second pad.

Furthermore, according to still another aspect of the present invention, it is desirable that the first pad is formed on a surface of the apparatus introduced inside the body.

According to still another aspect of the present invention, it is desirable that the apparatus introduced inside the body is a medical capsule device which includes an outer covering having a cylindrical shape with a base, and which can be introduced inside the body, and the first pad is provided on a surface of the medical capsule device.

Moreover, according to still another aspect of the present invention, it is desirable that

at least the image signal is transmitted from the medical capsule device to the apparatus outside the body, and

at least an electric power for driving the medical capsule device is transmitted from the apparatus outside the body to the medical capsule device.

### BRIEF DESCRIPTION OF THE DRAWINGS

FIG. 1 is diagram showing an overall structure of an in-body information acquisition system according to a first embodiment of the present invention;

FIG. 2 is a diagram showing an external structure of a medical capsule device in the first embodiment;

FIG. 3 is a functional block diagram of the medical capsule device of the first embodiment;

FIG. 4 is a functional block diagram of an in vitro apparatus of the first embodiment;

FIG. 5 is a diagram showing a cross-sectional structure of a pad of the in vitro apparatus of the first embodiment;

FIG. 6 is a functional block diagram a medical capsule device of a second embodiment;

FIG. 7 is a functional block diagram of an in vitro apparatus of the second invention;

FIG. 8 is a flowchart showing a flow of a signal in the second embodiment; and

FIG. 9 is another flowchart showing the flow of a signal in the second embodiment.

### DETAILED DESCRIPTION OF THE PREFERRED EMBODIMENTS

Embodiments of an in-body information acquisition system according to the present invention will be described below in detail while referring to the accompanying diagrams. However, the present invention is not restricted to these embodiments.

### First Embodiment

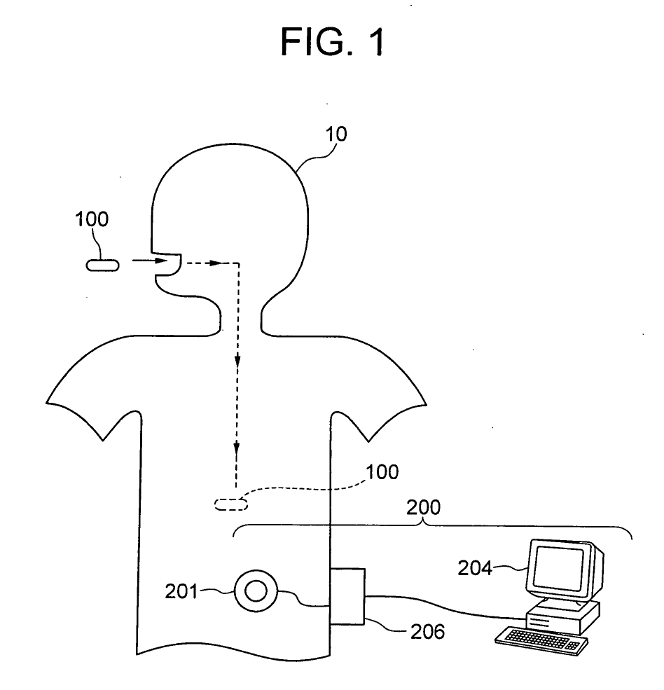

FIG. 1 is a diagram showing a schematic structure of an in-body information acquisition system according to a first embodiment of the present invention. In FIG. 1, a body 10 which is a living body to be examined, and a case of acquiring in vivo information of a patient for example, are shown. A medical capsule device 100 such as a capsule endoscope has a function of moving with a peristaltic motion inside an organ such as a stomach and a small intestine and take images one after another, during an observation time from being swallowed for observation (examination) from a mouth by a patient till discharged out naturally from the body.

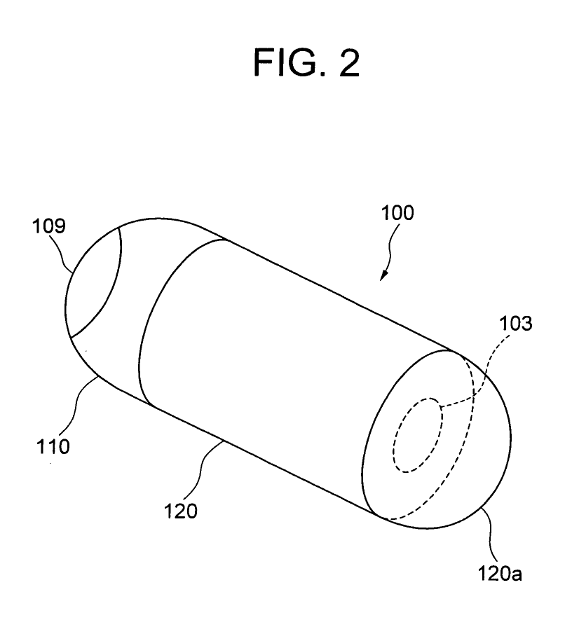

FIG. 2 shows a schematic external structure of the medical capsule device 100. The medical capsule device 100 corresponds to the apparatus introduced inside the body. Moreover, the medical capsule device 100 includes an outer covering 120 having a cylindrical shape with a base, and which can be introduced in the body 10. Furthermore, a first pad 109 which will be described later is formed on a surface of the medical capsule device 100. A CCD (charge coupled device) 103 is formed on a side opposite to a side on which the first pad 109 is formed. A third pad 110 having an annular shape (ring shape) will be described in a second embodiment.

Image data which is taken in the body by the medical capsule device 100 during observation by the movement in the organ is transmitted one after another to an apparatus outside the body (hereinafter, “in vitro apparatus”) 200 by a communication means which will be described later. The medical capsule device 100 and the in vitro apparatus 200 form the in-body information acquisition system. First of all, a structure of the medical capsule device 100 will be described, and then a structure of the in vitro apparatus 200 will be described.

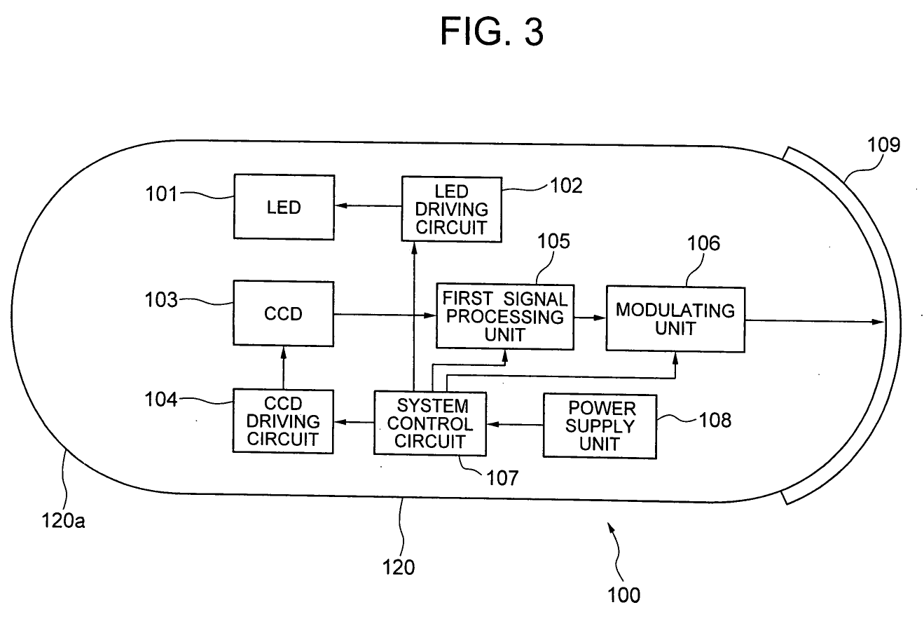

FIG. 3 is a functional block diagram of the medical capsule device 100. The medical capsule device 100 includes an LED (light emitting diode) 101 for illuminating an imaging area at the time of imaging inside the body 10, an LED driving circuit 102 which controls a driving of the LED 101, and a CCD 103 which takes images of the area illuminated in the body by the LED 101. Moreover, the medical capsule device 100 includes a CCD driving circuit 104, a first signal processing unit 105, a modulating unit 106, the first pad 109, and a system control circuit 107. The CCD driving circuit 104 controls a driving of the CCD 103. The first signal processing unit 105 processes image data (image signal) taken by the CCD 103. The modulating unit 106 modulates an in vivo information signal from the first signal processing unit 105. A voltage modulated from the modulating unit 105 is applied to the first pad 109. The system control circuit 107 controls an operation of each of the LED driving circuit 102, the CCD driving circuit 104, the first signal processing unit 105, and the modulating unit 106. Moreover, a power supply unit 108 supplies an electric power to each unit and circuit etc. in the medical capsule device 100.

The CCD 103 acquires in vivo information such as image information inside the body 10. The CCD 103 corresponds to the imaging section, and has a function as an in vivo information sensor. Apart from the CCD 103, CMOS (complementary metal oxide semiconductor) can be used as the imaging section. At least a part of a window 120a on the outer covering of the medical capsule device 100 is formed of a material such as a transparent material. The CCD 103 takes an image of (inside of) the body through the window 120a.

The CCD 103 is connected to the CCD driving circuit 104. The CCD driving circuit 104 outputs to the CCD 103 an actuating signal (operation signal) for acquiring the in vivo information. The CCD 103 is connected to the first signal processing unit 105. The first signal processing unit 105 has a function as an in vivo information processing unit. The first signal processing unit 105 includes circuits such as a data compression circuit and an image converting circuit for output from the CCD 103. Moreover, the first signal processing unit 105 generates an in vivo information signal from an output signal of the CCD 103, and outputs the in vivo information signal which is generated.

The CCD driving circuit 104 and the first signal processing unit 105 are connected to the modulating unit 106 via the system control circuit 107. The modulating unit 106 modulates the output signal from the first signal processing unit 105, and applies a voltage to the first pad 109.

The first pad 109 is formed of a material such as copper (Cu) and nickel (Ni), which does not include any substance harmful to the body. In general, the first pad 109 is formed of a material such as platinum (Pt) and gold (Au).

The first pad 109 is formed on an outer surface of the medical capsule device 100. An inside of the medical capsule device 100 is a sealed structure. The first pad 109 is connected to the modulating unit 106 while maintaining the sealed state of the medical capsule device 100. The first pad 109 and the modulating unit 106 are formed by sealing a through hole by filling a material such as a resin and a metal, upon being connected by passing through the through hole (not shown in the diagram) of the medical capsule device 100. Next, the in vitro apparatus 200 will be described.

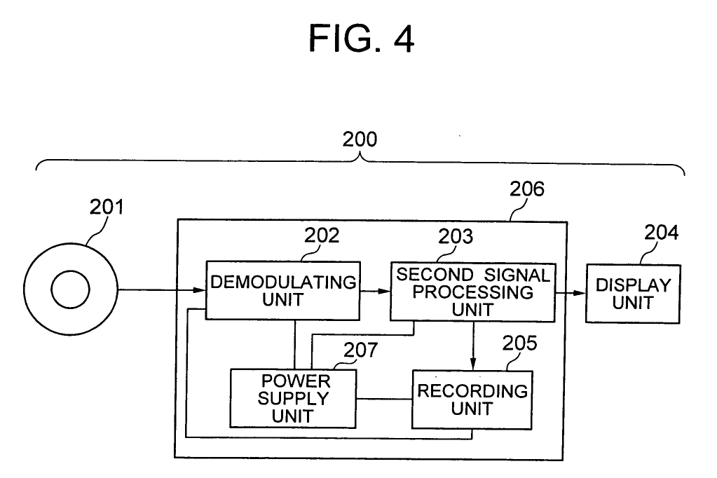

FIG. 4 is a functional block diagram of the in vitro apparatus 200. A second pad 201 is installed on the surface of the body 10. Moreover, the second pad 201 is connected to a demodulating unit 202 in a portable unit 206. The portable unit 206 is mounted near a waist belt of the body 10 for example.

The portable unit 206 includes the demodulating unit 202, a second signal processing unit 203, a recording unit 205, and a power supply unit 207. The demodulating unit 202 demodulates the output signal from the first signal processing unit 105 based on a change in an electric potential of a surface of the second pad 201.

By applying to the first pad 105 a voltage in which the output signal from the first signal processing unit 105 is modulated, there occurs to be a change in the electric potential on the surface of the second pad 201. The demodulating unit 202 demodulates the output signal from the first signal processing unit 105. Accordingly, a communication from an inside to the outside of the body 10 can be realized.

The second pad 201 is formed of a material such as copper (Cu) and nickel (Ni), which does not include any substance harmful to the body. In general, the second pad 201 is formed of a material such as platinum (Pt) and gold (Au).

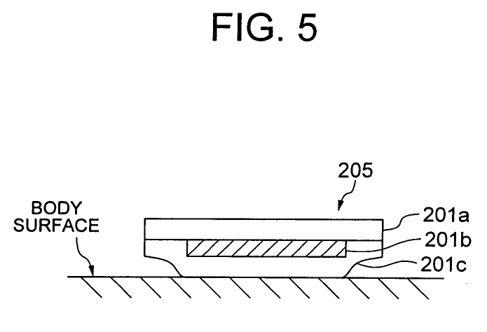

FIG. 5 shows a cross-sectional structure of the second pad 201. Since the second pad 201 makes a close contact with the body surface, the second pad 201 has a structure in which a thin film 201b made of platinum (Pt) and gold (Au) is sandwiched by a substrate 201 such as a resin film and a ribbon. Furthermore, a portion which makes a contact with the body surface is formed of an insulating thin-film 201c made of a material such as a silicon resin. It is desirable that a thickness of the insulating thin-film 201c is not greater than 1 mm such that the electric potential on the body surface can be detected at the second signal processing unit 203. Moreover, a gel or oil may be applied between the surface of the body 10 and the second pad 201. Accordingly, an adhesion between the second pad 201 and the body surface can be improved.

Thus, in the first embodiment, since an information communication which is independent of an electric current is performed, the second pad 201 can be let to have an insulating structure. Therefore, a safety of the body 10 can be improved.

The description will be continued upon coming back to FIG. 4. The demodulating unit 202 is connected to the second signal processing unit 203. The second signal processing unit 203 is a circuit such as a decompression circuit for compressed data, and correction/enhancing circuit of the image information. The second signal processing unit 203 performs a signal processing for acquiring the required in vivo information, based on the output signal from the first signal processing unit 105 which is demodulated by the demodulating unit 202.

Moreover, the second signal processing unit 203 is connected to a display unit 204. The display unit 204 is a monitor such as a liquid crystal display. The display unit 204 displays the in vivo information which is processed in the second signal processing unit 203. In FIG. 1, the display unit 204 is not provided on the portable unit 206 but provided elsewhere. However, without restricting to the structure in which the display unit 204 is not provided on the portable unit 206, the structure may be such that the display unit 204 is provided on the portable unit 206.

The recording unit 205 is connected to the demodulating unit 202 or to the second signal processing unit 203. The recording unit 205 includes a memory such as a semiconductor memory. The recording unit 205 records and stores the output signal from the first signal processing unit 105 which is demodulated by the demodulating unit 202 or the in vivo information which is processed in the second signal processing unit 203.

The power supply unit 207 supplies the electric power to the demodulating unit 202, the second signal processing unit 203, and the recording unit 205.

According to the first embodiment, the medical capsule device 100 and the in vitro apparatus 200 can communicate the in vivo information to the outside of the body independent of electric waves and electric current. Inventors of the present invention have been considering that the information can be communicated by electrostatic induction. The inventors made a practical apparatus, and tested and confirmed that such communication is possible.

Thus, in the first embodiment, a size of the medical capsule device 100 and the in vitro apparatus 200 is not required to be increased by installing a respective antenna and a transmitting circuit. Therefore, it is possible to provide a small size in-body information acquisition system which enables to reduce a strain on the body 10 of a patient.

### Second Embodiment

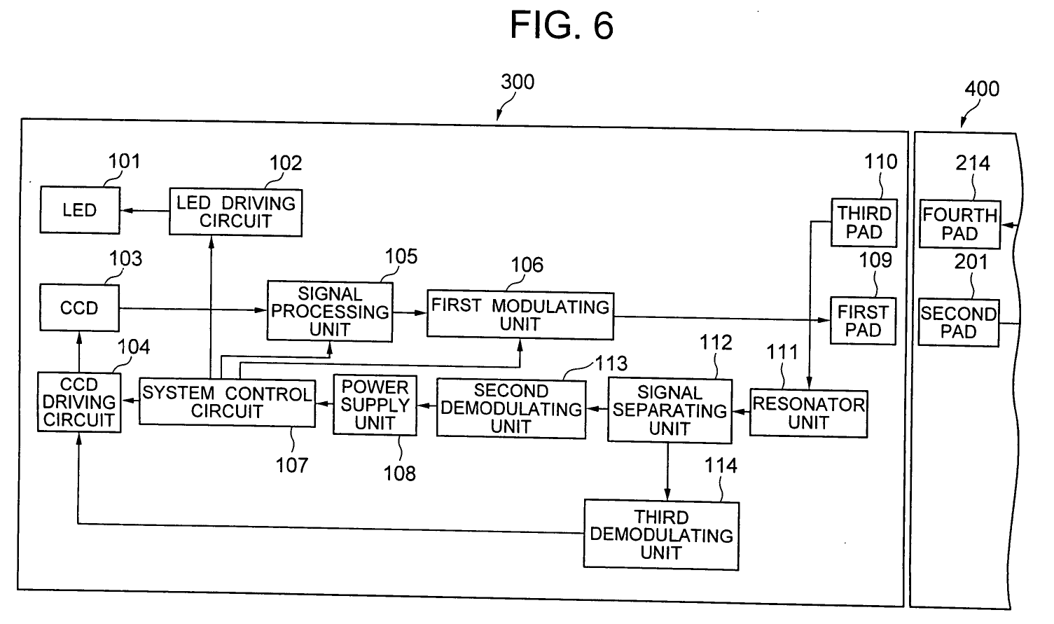

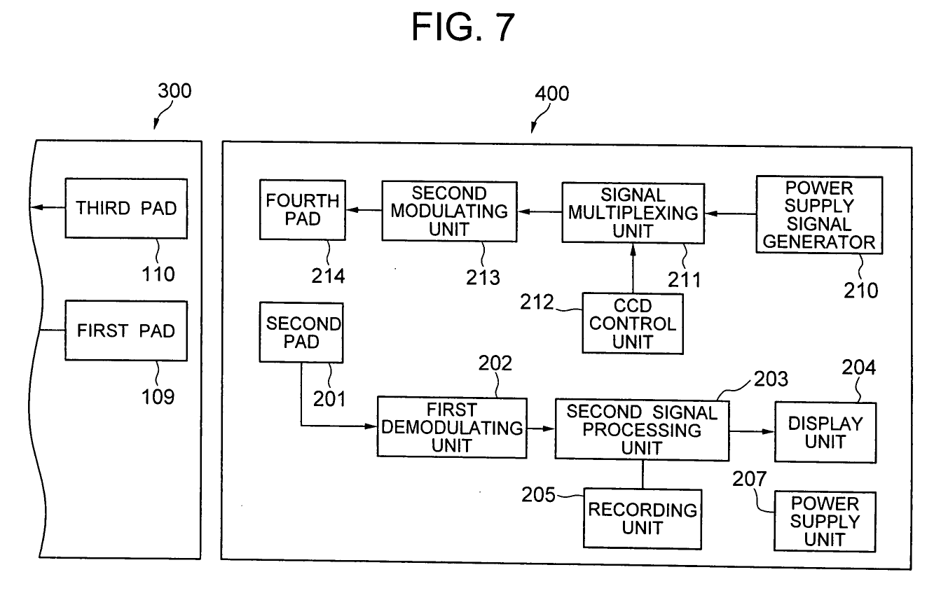

FIG. 6 is a functional block diagram of a medical capsule device 300 in a second embodiment of the present invention. Moreover, FIG. 7 is a functional block diagram of in vitro apparatus 400 in the second embodiment. The medical capsule device 300 and the in vitro apparatus 400 form an in-body information acquisition system. In the second embodiment, same reference numerals are assigned to components which are same as in the first embodiment, and the description to be repeated is omitted.

The second embodiment differs from the first embodiment at a point that apart from communicating the image data from the medical capsule device 300 to the in vitro apparatus 400, the power supply and control signals are also communicated (transmitted) from the in vitro apparatus 400 to the medical capsule device 300.

In the second embodiment, the first pad 109 formed on a side of the medical capsule device 300 and the second pad 201 formed on a side of the in vitro apparatus 400 are disposed at positions facing mutually, to be coupled electrostatically. Similarly, a third pad 110 formed on the side of the medical capsule device 300 and a fourth pad 214 formed on the side of the in vitro apparatus 400 are disposed at positions facing mutually, to be coupled electrostatically.

Moreover, in the second embodiment, as shown in FIG. 2, an electric conductor which forms the annular shaped (ring shaped) third pad 110 is provided on an outer circumference of an electric conductor which forms the first pad 109. However, without restricting to such structure, other structure such as a structure in which the first pad 109 and the third pad 110 are disposed side by side, can also be adopted.

Furthermore, a structure which enables to serve the purpose by one electric conductor can also be adopted. In such structure, by using a different modulation frequency for each of a first modulating unit 106 on the side of the medical capsule device 300 and a second modulating unit 213 on the side of the in vitro apparatus 400, only one pad can serve as the first pad 109 and the third pad 110.

Moreover, similarly as in the first embodiment, a voltage in which an output of a signal processing unit 105 is modulated, is applied to the first pad 109. Based on the change in the electric potential of the surface of the second pad 201 occurred due to applying the voltage, a first demodulating unit 202 demodulates the output signal from the signal processing unit 105. Accordingly, it is possible to communicate (transmit) signals such as an image signal from the medical capsule device 300 to the in vitro apparatus 400.

Next, the communication of a signal from the in vitro apparatus 400 to the medical capsule device 300 will be described. In FIG. 7, the in vitro apparatus 400 includes a power supply signal generator 210, a CCD control unit 212, and a signal multiplexing unit 211. The power supply signal generator 210 outputs a power supply voltage signal of a predetermined frequency. The CCD control unit 212 outputs a control signal to the CCD 103 such as a control signal for CCD sensitivity.

The signal multiplexing unit 211 superimposes the control signal output from the CCD control unit 212 to the CCD 103, on a voltage signal which is output from the power supply signal generator 210. The signal multiplexing unit 211 is connected to the second modulating unit 213. Moreover, the second modulating unit 213 is connected to the fourth pad 214. The second modulating unit 213 modulates an output signal from the signal multiplexing unit 211, and applies the voltage to the fourth pad 214.

Next, the description will be continued upon coming back to FIG. 6. The third pad 110 is connected to a resonator unit 111 which is provided inside the medical capsule device 300. The resonator unit 111 outputs upon extracting a frequency component which is modulated by the second modulating unit 213 based on the change in the electric potential of the third pad 110, due to an electrical resonance.

The resonator unit 111 is connected to a signal separating unit 112. The signal separating unit 112 is connected to a second demodulating unit 113 and a third demodulating unit 114.

The signal separating unit 112 separates the change in the electric potential of the third pad 110 which is output upon extracting by the resonator unit 111, into a voltage signal component, and a control signal component to the CCD 103. Moreover, the signal separating unit 112 outputs the power supply voltage signal component to the second demodulating unit 113. Furthermore, the signal separating unit 112 outputs the control signal component to the CCD 103, to the third demodulating unit 114.

The second demodulating unit 113 demodulates a voltage signal output from the power supply signal generator 210, based on the voltage signal component of the change in the potential of the third pad 110, which is output from the signal separating unit 112.

The second modulating unit 113 is connected to the power supply unit 108. The power supply unit 108 supplies power for operating each unit and circuit in the medical capsule device 300, from the voltage signal demodulated by the second demodulating unit 113 via the system control circuit 108.

Thus, the voltage in which a signal on which the control signal to the CCD 103 which is output by the CCD control unit 212 is superimposed, is demodulated, is applied to the voltage signal which is output to the fourth pad 214 by the power supply signal generator 210. Moreover, on the side of the medical capsule device 300, the voltage signal output by the power supply signal generator 210 is demodulated upon separating from the change in the electric potential of a surface of the third pad 110 which has occurred due to applying the voltage. Accordingly, it is possible to supply the electric power from the in vitro apparatus 400 to the medical capsule device 300. As a result, in the in-body information acquisition system of the second embodiment, even when compared to a power supply by an electromagnetic induction, the size of the system is not increased due to a winding etc. Moreover, it is possible to realize an airtight and watertight structure which is necessary in the medical capsule device 300.

Furthermore, the third demodulating unit 114 demodulates the control signal of the CCD 113 which is output by the CCD control unit 212, based on the voltage signal component of the change in the potential of the third pad 110 which is output by the signal separating unit 112.

The third demodulating unit 114 is connected to the CCD driving circuit 104. The CCD 103 is driven based on the control signal to the CCD 103 from the CCD control unit 212 which is demodulated, such as an instruction signal of sensitivity control.

Thus, the voltage in which the signal on which the control signal to the CCD 103 is output by the CCD control unit 212 is superimposed, is demodulated, is applied to the voltage signal which is output to the fourth pad 214 by the power supply signal generator 210. Moreover, on the side of the medical capsule device 300, the voltage signal output by the CCD control unit 212 to the CCD 103 is demodulated upon separating from the change in the electric potential of the surface of the third pad which has occurred due to applying the voltage. Accordingly, it is possible to realize a signal communication from the in vitro apparatus 400 to the medical capsule device 300. As a result, in the in-body information acquisition system of the second embodiment, the size of the system is not increased due to installing an antenna for transceiving (transmitting and/or receiving) the electric waves. Moreover, it is possible to realize an airtight and watertight structure which is necessary in the medical capsule device 300.

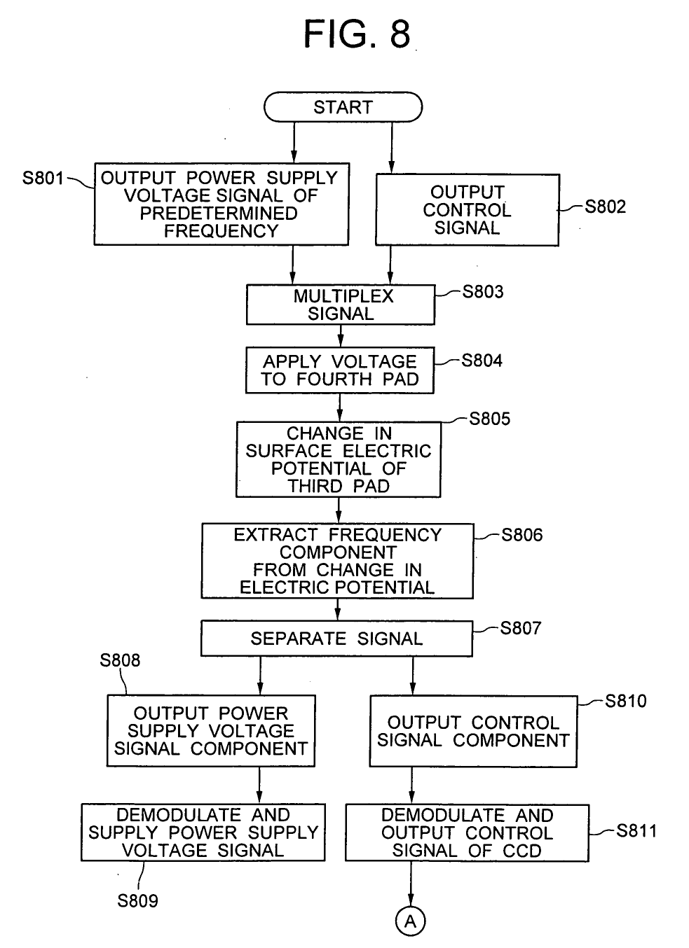

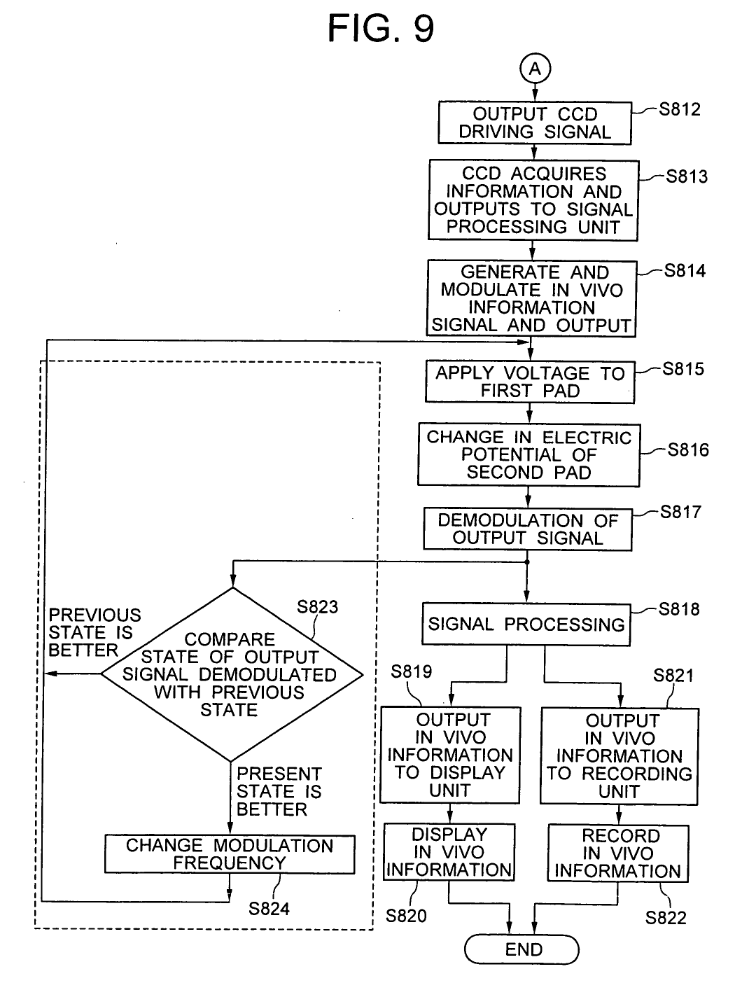

Next, a flow of a signal in the second embodiment will be described in further detail, with reference to flowcharts. Each of FIG. 8 and FIG. 9 is a flowchart showing the flow of the signal in the second embodiment.

At step S801, the power supply signal generator 210 outputs a power supply voltage signal of a predetermined frequency to the signal multiplexing unit 211. At step S802, the CCD control unit 212 outputs to the signal multiplexing unit 211, a control signal to the CCD 103.

At step S803, the signal multiplexing unit 211 superimposes the control signal to the CCD 103 which is output by the CCD control unit 212, on the voltage signal which is output by the power supply signal generator 210, and outputs to the second modulating unit 213.

At step S804, the second modulating unit 213 demodulates the output signal of the signal multiplexing unit 211, and applies voltage to the fourth pad 214. At step S805, the electric potential of the surface of the third pad 110 is changed due to the voltage applied to the fourth pad 214 which has modulated the output signal of the signal multiplexing unit 211.

At step S806, the resonator unit 111 extracts a frequency component which is output upon modulating by the second modulating unit 213 from the change in the electric potential of the third pad 110 by the electrical resonance.

At step S807, the signal separating unit 112 separates the change in the electric potential of the third pad 110 which is extracted by the resonator unit 111, into a power supply voltage signal component, and a control signal component to the CCD 103.

At step S808, the signal separating unit 112 outputs the power supply voltage signal component separated by the signal separating unit 112 to the second demodulating unit 113. At step S809, the second demodulating unit 113 demodulates a power supply voltage signal output to the power supply signal generator 210, based on the change in the electric potential of the third pad 110. Further, the power supply voltage signal (electric power) which is modulated is supplied to each unit and each circuit etc. in the medical capsule device 300 via the power supply unit 108.

At step S810, the signal separating unit 112 outputs to the third demodulating unit 114, the control signal component to the CCD 103. At step S811, the third demodulating unit 114 demodulates the control signal to the CCD 103 which is output by the CCD control unit 212, based on the change in the electric potential of the third pad 110. Further, the third demodulating unit 114 outputs the control signal demodulated, to the CCD driving circuit 104.

Next, at step S812 in FIG. 9, the CCD driving circuit 104 outputs a driving signal to the CCD 103. At step S813, the CCD 103 acquires (images) in vivo information. Further, the CCD 103 outputs the in vivo information which is acquired, to the signal processing unit 105.

At step S814, the signal processing unit 105 generates an in vivo information signal based on the output signal of the CCD 103. Further, the signal processing unit 105 outputs the in vivo information signal generated, to the first modulating unit 106.

At step S815, the first modulating unit 106 modulates the output signal from the signal processing unit 105. Further, the first modulating unit 106 applies voltage to the first pad 109 corresponding to the output signal which is modulated.

At step S816, the electric potential of the surface of the second pad 201 is changed due to the voltage applied to the first pad, in which the output signal from the signal processing unit 105 is modulated. At step S817, the first demodulating unit 202 demodulates the output signal of the signal processing unit 105, based on the change in the electric potential of the surface of the second pad 201. Further, the first demodulating unit 202 outputs the output signal demodulated to the second signal processing unit 203.

At step S818, the second signal processing unit 203 performs a signal processing for acquiring the required in vivo information, from the output signal of the signal processing unit 105 which is demodulated by the first demodulating unit 202.

At step S819, the second signal processing unit 203 outputs the in vivo information acquired during the signal processing, to the display unit 204. At step S820, the display unit 204 displays the in vivo information.

At step S821, the second signal processing unit 203 outputs the in vivo information acquired during the signal processing, to the recording unit 205. At step s822, the recording unit 205 records and stores the in vivo information.

Next, an optimization of the modulation frequency will be described. Based on a state (S/N ratio) of the output signal from the second signal processing unit, which is demodulated at the first demodulating unit 202, it is possible to determine a modulation frequency when the first modulating unit 106 modulates the output signal of the signal processing unit 105, and applies voltage to the first pad 109.

For example, with an initial demodulation frequency by the first modulating unit 106 as a base (reference), the modulation frequency is changed to a lower side and a higher side of the initial modulation frequency. The initial modulation frequency means a frequency determined by experiment etc. at which a state of the output signal of the second signal processing unit 203 is favorable in general.

Moreover, the state of the output signal of the second signal processing unit 203 which is demodulated by the first demodulating unit 202, for example a frequency at which the S/N ratio for example, becomes favorable, is determined to be the optimum frequency.

Moreover, regarding the change of the modulation frequency, the frequency to be changed may be determined randomly, or the modulation frequency may be adjusted promptly to be the optimum modulation frequency by also using a so-called mountain climbing method (method of steepest gradient). Apart from this, the frequency to be changed can be determined by using any algorithm.

A procedure for determining the optimum frequency in such manner will be described by referring to the flowchart in FIG. 9. At step S823, subsequent to the step S817, the state (S/N ratio) of the output signal of the second signal processing unit 203 which is demodulated by the first demodulating unit 202 is compared with a previous state. When the present state is better than the previous state, at step S824, the modulation frequency is changed to the frequency at present. Next, the process returns to step S815. When the previous state is better than the present state, the process returns to step S815. Thus, in FIG. 9, procedure enclosed by dotted lines corresponds to an optimization procedure of the modulation frequency.

Accordingly, it is possible reduce an effect of an individual variation of the body 10 and a difference in a state of the body 10 according to that time and date, and to realize even more favorable communication between the medical capsule device 300 and the in vitro apparatus 400.

Moreover, a medical capsule device in each of the embodiments is structured to take an image of the inside by providing an LED and a CCD. However, an in vivo apparatus which is introduced in the body is not restricted to such structure, and may be let to be an apparatus which acquires other in vivo information such as information of temperature and pH of the body.

Moreover, the present invention is not restricted to a medical capsule device which is to be swallowed, and can be applied to a normal endoscope which is inserted into the body. In this case, it is possible to communicate easily in vivo information such as a temperature to an outside by this system, and to improve the airtightness of the endoscope. Moreover, the present invention can also be applied to a so-called cardiac pacemaker. For example, by this system, information for driving the pacemaker can be communicated to the pacemaker from the outside. Furthermore, information such as history information recorded in the pacemaker can be communicated to the outside without exerting strain on a person wearing the pacemaker.

Moreover, in the first embodiment and the second embodiment, as a body to be examined, an example of examining and observing a human body is shown. However, the present invention is not restricted to examining the human body only, and an industrial product for example, may be used as a body to be examined.

In the in-body information acquisition system according to the present invention, between a first pad of an in vivo apparatus and a second pad of an in vitro apparatus, a voltage is applied upon modulating a signal, to the pad in one of the in vivo apparatus and the in vitro apparatus. Moreover, in the other apparatus, the signal is demodulated from a change in a potential difference of the pad. Accordingly, it is possible to communicate information without using electric waves and electric current, between the in vivo apparatus and the in vitro apparatus. Therefore, when the information is to be communicated from the in vivo apparatus to the in vitro apparatus, the in vivo apparatus is not required to have an antenna and a transmitting circuit, and consequently it is possible to reduce a size of the in vivo apparatus. Moreover, also regarding the in vitro apparatus, a structure in which a plurality of antennas for receiving a signal is disposed near a body, such as a body of a patient, and a detection of a weak current, and a demodulating circuit are not required. Consequently, the in vivo apparatus and the in vitro apparatus are not required to be large scale by installing the antenna etc. As a result, it is possible to provide an in-body information acquisition system having a small size, and which enables to reduce a strain on (the body of) the patient.

Thus, the in-body information acquisition system of the present invention is useful as a small size system for reducing the strain on (the body of) the patient.

What is claimed is:

1. An in-body information acquisition system comprising:
an apparatus introduced inside a body which is introduced inside the body; and
an apparatus outside the body which is disposed outside the body, and which communicates with the apparatus introduced inside the body, wherein
the apparatus introduced inside the body includes at least a first pad, and
the apparatus outside the body includes at least a second pad, wherein
for transceiving a signal between the first pad and the second pad, at least one of the apparatus introduced inside the body and the apparatus outside the body includes a modulating unit which modulates a signal and applies a voltage to the pad of one of the apparatus introduced inside the body and the apparatus outside the body, and
the other apparatus includes a demodulating unit which demodulates the signal based on a change in an electric potential of the pad of the other apparatus.

2. The in-body information acquisition system according to claim 1, wherein
the apparatus introduced inside the body includes an imaging section which takes an image of a part of the body to be examined, and outputs at least an image signal, and
the apparatus outside the body demodulates the image signal.

3. The in-body information acquisition system according to claim 2, wherein
the second pad of the apparatus outside the body is disposed to be in contact with a surface of the body.

4. The in-body information acquisition system according to claim 3, wherein
an insulating member is formed on a surface of at least one of the first pad and the second pad.

5. The in-body information acquisition system according to claim 4, wherein
the first pad is formed on a surface of the apparatus introduced inside the body.

6. The in-body information acquisition system according to claim 3, wherein
the first pad is formed on a surface of the apparatus introduced inside the body.

7. The in-body information acquisition system according to claim 3, wherein
an insulating member is formed on a surface of at least on one of the first pad and the second pad.

8. The in-body information acquisition system according to claim 7, wherein
the first pad is formed on a surface of the apparatus introduced inside the body.

9. The in-body information acquisition system according to claim 2, wherein
the first pad is formed on a surface of the apparatus introduced inside the body.

10. The in-body information acquisition system according to claim 1, wherein
the second pad of the apparatus outside the body is disposed to be in contact with a surface of the body.

11. The in-body information acquisition system according to claim 10, wherein
an insulating member is formed on a surface of at least one of the first pad and the second pad.

12. The in-body information acquisition system according to claim 11, wherein
the first pad is formed on a surface of the apparatus introduced inside the body.

13. The in-body information acquisition system according to claim 10, wherein
the first pad is formed on a surface of the apparatus introduced inside the body.

14. The in-body information acquisition system according to claim 1, wherein
an insulating member is formed on a surface of at least one of the first pad and the second pad.

15. The in-body information acquisition system according to claim 14, wherein
the first pad is formed on a surface of the apparatus introduced inside the body.

16. The in-body information acquisition system according to claim 1, wherein
the first pad is formed on a surface of the apparatus introduced inside the body.

17. The in-body information acquisition system according to claim 1, wherein
the apparatus introduced inside the body is a medical capsule device which includes an outer covering having a cylindrical shape with a base, and which can be introduced inside the body, and
the first pad is formed on a surface of the medical capsule device.

18. The in-body information acquisition apparatus according to claim 17, wherein
at least an image signal is transmitted from the medical capsule device to the apparatus outside the body, and
at least an electric power for driving the medical capsule device is transmitted from the apparatus outside the body to the medical capsule device.
